(19) United States
(12) Patent Application Publication
(10) Pub. No.: US 2010/0120118 A1
(43) Pub. Date: May 13, 2010

(54) MRNA Interferase from Myxococcus Xanthus

(75) Inventors: Masayori Inouye, New Brunswick, NJ (US); Hirofumi Nariya, Highland Park, NJ (US)

Correspondence Address: PATENT DOCKET ADMINISTRATOR;LOWENSTEIN SANDLER PC, 65 LIVINGSTON AVENUE, ROSELAND, NJ, 07068, US

(73) Assignee: University of Medicine and Dentistry of New Jersey, Somerset, NJ (US)

(21) Appl. No.: 12/593,549

(22) PCT Filed: Mar. 28, 2008
(86) PCT No.: PCT/US08/58737

§ 371 Date: Dec. 22, 2009

Related U.S. Application Data
(60) Provisional application No. 60/920,476, filed on Mar. 28, 2007.

(51) Int. Cl. C12N 9/99; C12N 1/20; C12N 9/22; C07H 21/00

(52) U.S. Cl. 435/184; 435/252.1; 435/199; 536/23.1

(57) ABSTRACT

A deployment of a toxin gene for developmental programmed cell death in bacteria is described. M. xanthus is demonstrated to have a solitary mazF gene that lacks a cotranscribed antitoxin gene. Deletion of mazF results in elimination of the obligatory cell death during development causing dramatic reduction in spore formation. Surprisingly, MrpC functions as a MazF antitoxin and a mazF transcription activator. Transcription of mrpC and mazF is negatively regulated via MrpC phosphorylation by a Ser/Thr kinase cascade. Various methods of exploiting this novel pathway are described herein.

### CLAIM OF PRIORITY

This application claims priority to U.S. Provisional Application No. 60/920,476, filed Mar. 28, 2007, the disclosure of which is hereby incorporated by reference in its entirety.

### STATEMENT REGARDING REFERENCES

All patents, publications, and non-patent references referred to herein shall be considered incorporated by reference into this application in their entireties.

### STATEMENT UNDER 37 C.F.R. §1.821(f)

In accordance with 37 C.F.R. §1.821(f), the content of the attached Sequence Listing and the attached computer readable copy of the Sequence Listing are identical.

### BACKGROUND OF THE INVENTION

While programmed cell death (“PCD”) pathway is a well-established eukaryotic developmental process, it has been unclear if any developmental pathways in bacteria similarly require a well-defined PCD pathway. Obligatory cell lysis during development observed during Bacillus sporulation and Myxobacteria fruiting body formation exemplify forms of bacterial PCD (K. Lewis, Microbial. Mol. Biol. Rev. 64, 503 (2006), H. Engelberg-Kulka, R. Hazan, Science 301, 467 (2003)). Myxococcus xanthus, a unique soil Gram-negative bacterium, exhibits social behavior during vegetative growth and multicellular development forming fruiting bodies upon nutrient starvation. The developmental processes of M. xanthus has been shown to be regulated by a series of sophisticated intercellular signaling pathways that activate expression of a different set of genes with precise temporal patterns during development (M. Dworkin, Microbial. Rev. 60, 70 (1996), B. Julien, A. D. Kaiser, A. Garza, Proc. Natl. Acad. Sci. U.S.A. 97, 9098 (2000)). During M. xanthus fruiting body formation, the majority (approximately 80%) of the cells undergo altruistic obligatory cell lysis, while the remaining 20% are converted to myxospores (J. W. Wireman, M. Dworkin, J. Bacterial. 29, 798 (1977), H. Nariya, S. Inouye, Mol. Microbiol. 49, 517 (2003)). Although the exact autolysis mechanism remains obscure, M. xanthus contains a large number of autolysin genes encoding for enzymes that degrade the cell wall (TIGR: http://cmr.tigr.org/tigr-scripts/CMR/GenomePage.cgi?org=gmx). Curiously, however, none of these autolysin genes have been shown to be essential for developmental autolysis.

The toxin-antitoxin (“TA”) systems are widely found in bacterial chromosomes and plasmids. These systems generally consist of an operon that encodes a stable toxin and its cognate labile antitoxin. Genomic analysis of 126 prokaryotes revealed that there are at least eleven genome-encoded TA systems (MazEF, RelEB, DinJ/YafQ, YefM/YeoB, ParDE, HigBA, VapBC, Phd/Doc, CcdAB, HipAB and εζ) in free-living bacteria, while obligate host-associated bacteria living in constant environmental condition do not possess the TA modules (V. S. Lioy et al., Microbiology 152, 2365 (2006), D. P. Pandey, K. Gerdes, Nucleic Acids Res. 33, 966 (2005)). This finding has allowed the suggestion that the TA systems may play important roles during adaptation to environmental stresses. Among the TA systems, the MazE-MazF system remains one of the best-studied systems; MazF from Escherichia coli has been shown to be an mRNA interferase specifically cleaving cellular mRNAs at ACA sequences to effectively inhibit protein synthesis and subsequent cell growth (Y. Zhang, J. Zhang, K. P. Hoeflich, M. Ikura, G. QingM. Inouye, Mol. Cell. 12, 913 (2003)). MazF induction in E. coli leads to a new physiological cellular state termed “quasidormancy,” under which cells are fully metabolically active and still capable of producing a protein in the complete absence of other cellular protein synthesis if the mRNA for the protein is engineered to have no ACA sequences (M. Suzuki, J. Zhang, M. Liu, N. A. Woychik, M. Inouye, Mol. Cell 18, 253. (2005)).

### SUMMARY OF THE INVENTION

Previously, a killing factor exported from sporulating bacterial cells (Bacillus subtilus) has been described, which cooperatively blocks sister cells from sporulation to cause them to lyse leading to cell death. The sporulating cells feed on the nutrients released from the lysed sister cells to complete spore formation. In contrast to such an extra-cellular death factor secreted from a selected population of sporulating bacterial cells, disclosed herein is a bacterial developmental PCD pathway regulated by a death factor in the cells that is reminiscent of eukaryotic PCD. In prokaryotes, the toxin-antitoxin (“TA”) systems play important roles in growth regulation under stress conditions. In the E. coli MazE-MazF system, MazF toxin functions as an mRNA interferase cleaving mRNAs at ACA sequences to effectively inhibit protein synthesis leading to cell growth arrest. Myxococcus xanthus is a Gram-negative bacterium displaying spectacular multi-cellular fruiting body development during which 80% of the cells undergo obligatory cell lysis upon the onset of development initiated by nutrient starvation. It has been found that this bacterium has a solitary mazF gene (mazF-mx) without its cognate antitoxin gene, mazE-mx, in contrast to other bacteria in which mazF encoding for an mRNA interferase, a sequence-specific endoribonuclease (E. coli MazF cleaves mRNAs at ACA sequences), is co-transcribed with its cognate antitoxin gene, mazE, in an operon. When the mazF-mx gene was deleted form the chromosome, the obligatory cell lysis during the fruiting body formation was eliminated causing dramatic reduction of spore formation. Surprisingly, MrpC, a key essential regulator for development, functions as a MazF-mx antitoxin fowling a stable complex, which also functions as a developmental transcription activator for mazF-mx to induce MazF-mx expression upon the onset of development. Further shown is that MazF-mx is an mRNA interferase recognizing a five-base sequence, GUUGC, to cleave between the two U residues, and that the antitoxin function of MrpC is regulated by a Ser/Thr protein kinase cascade.

These findings uncover for the first time the existence of a sophisticated PCD cascade associated with protein SerfThr kinases even in bacteria, which undergo multi-cellular development accompanying obligatory cell death (H. Nariya and M. Inouye, Cell 132, 55-66, Jan. 11, 2008).

In certain embodiments, the present invention is directed to inhibiting MazF-mx endoribonuclease activity by pre-incubating MazF-mx with MrpC.

In other embodiments, the present invention is directed to the use of MrpC as an antitoxin for MazF-mx.

In further embodiments, the invention is directed to reducing spore formation of Myxococcus xanthus by inactivating the mazF-mx gene.

In other embodiments, this invention is directed to inhibiting cell lysis of Myxococcus xanthus by inactivating the mazF-mx gene.

In further embodiments, this invention is directed to an isolated mazF-mx polypeptide.

In other embodiments, this invention is directed to a polynucleotide encoding the MazF-mx polypeptide.

In further embodiments, this invention is directed to a polynucleotide that hybridizes to the complement strand of the mazF-mx polynucleotide in stringent conditions.

In other embodiments, this invention is directed to the promoter region of mazF-mx as disclosed in FIG. 6.

In further embodiments, this invention is directed to producing polypeptides having endoribonuclease activity by transforming a host via introduction of a mazF-mx polynucleotide and culturing the transformed host.

### BRIEF DESCRIPTION OF THE DRAWINGS

FIG. 1. A. Interaction between MazF-mx and MrpC in a pull-down assay. Soluble fraction (S) from E. coli cells expressing non-tagged MazF-mx was incubated with (+) or without (−) purified His-tagged MrpC. The complex was recovered by the nickel-resin. The positions of His-tagged MrpC and MazF-mx are shown by arrows. B. Developmental phenotypes on CF agar plates after 12, 24, 36 and 48 h after development. Spore yields at 36 and 48 h are shown as taking the yield of the wild-type DZF 1 at 48 h as 100%. C and D. Developmental analysis of the total cell numbers and colony forming units (CFU). Numbers of rod-shape cells (solid line) and CFU (dotted line) of ΔmazF (open circles), DZF1 (closed circles) and ΔmrpC (open squares) were measured in C. The ratios of CFU to cell number were plotted in D.

FIG. 2. Expression and regulation of the mazF-mx gene during the M. xanthus life cycles. A. Primer-extension analysis of the mazF-mx expression after development. B. β-galactosidase assay of mazFmx promoter lacZ fusion integrated into the chromosome. C. Gel-shift assay of MrpC on the mazFmx promoter. D. Gel-shift assay of MrpC preincubated with purified His-tagged MazF-mx (H-MazF) prior to gel-shift assay. E. Primer-extension analysis for mazF-mx expression using total RNA from the wild-type (DZF1) and ΔmrpC cells at 0, 12 and 24 h after development.

FIG. 3. A. Cell toxicity of MazF-mx expression during vegetative growth in ΔmazF and ΔmrpC. These cells were transformed with either pKSAT-MazF-mx or pKSAT; pKSAT (filled circles) or pKSAT-HA-MazF-mx (open circles) in ΔmazF (solid lines) and pKSAT (filled squares) and pKSAT-HA-MazF-mx (open squares) in ΔmrpC (dotted lines). B. Development morphology on CF agar plates and spore yields at 48 h after development. The spore yield is a percentage of that for DZF1. C. Constitutive expression of pKSAT-HA-MazF-mx in ΔmrpC at the mid-log (16.5 h; lane 1) and mid-stationary (48 h; lane 2) phase during vegetative growth detected HA antibody. MazF-mx expression in the ΔmazF cells carrying pKSAT-MazF-mx at 16.5 h (lane 3) in vegetative growth in A.

FIG. 4. Endoribonuclease activity of MazF-mx in vitro. A. Cleavage of M. xanthus total RNA by His-tagged(H)-MazF. The products were 5′-end labeled with [γ-³²P]-ATP by T4 kinase and separated on agarose gel. The gel was stained with ethidium bromide (EtBr) and the dried gel was subjected to autoradiography. B and C. Cleavage of MS2 ssRNA and its inhibition by the antitoxin activity of MrpC. The gel vas stained with EtBr. D. Cleavage of 5′-end labeled MS2-0724-14 and the effect of phosphorylation of MrpC by Pkn14 on its antitoxin function. H-MazF was incubated with Pkn14 and Pkn14K48N (KN) in the presence of ATP. After dialysis, samples were examined their endoribonuclease activities. The products separated by 20% PAGE and subjected to autoradiography. The MS2-0724-14 and cleaved product were indicated by arrows.

FIG. 5. Sequence alignment of MazF homologs (A) and phylogenetic tree analysis of MazF (B). A. Alignment of M. xanthus MazF (Mx-MazF) with those of B. subtilis 168 (Bs), C. perfringens 13 (Cp), S. aureus COL (Sa), Nostoc PCC7120 (No), Synechocystis PCC6803 (Sy), M. tuberculosis H37Rv (Mt1˜7) and E. coli K12 (a). The gene symbols and locus tags are indicated (see also Table S2). β-strand (5) and helical (H) regions are assigned according to Ec-MazF. Amino acid residues identical are shown by black shades, and conservative substitutions by gray shades. Plasmid-borne MazF is indicated with an asterisk. B. Phylogenetic tree of MazF homologs was built by the neighbor joining method (http://crick.genes.nig.ac.jp) and illustrated by Tree View programs (http://taxonomy.zoology.gla.ac.uk) using the alignment shown in A.

FIG. 6. DNA sequence of the mazF promoter region. The transcription initiation site is indicated by +1. Putative MrpC binding sites, MazF1 and MazF2 are shown by bold letters. The sequences corresponding to primers used for PCR and the primer extension are underlined with arrows.

### DETAILED DESCRIPTION OF THE INVENTION

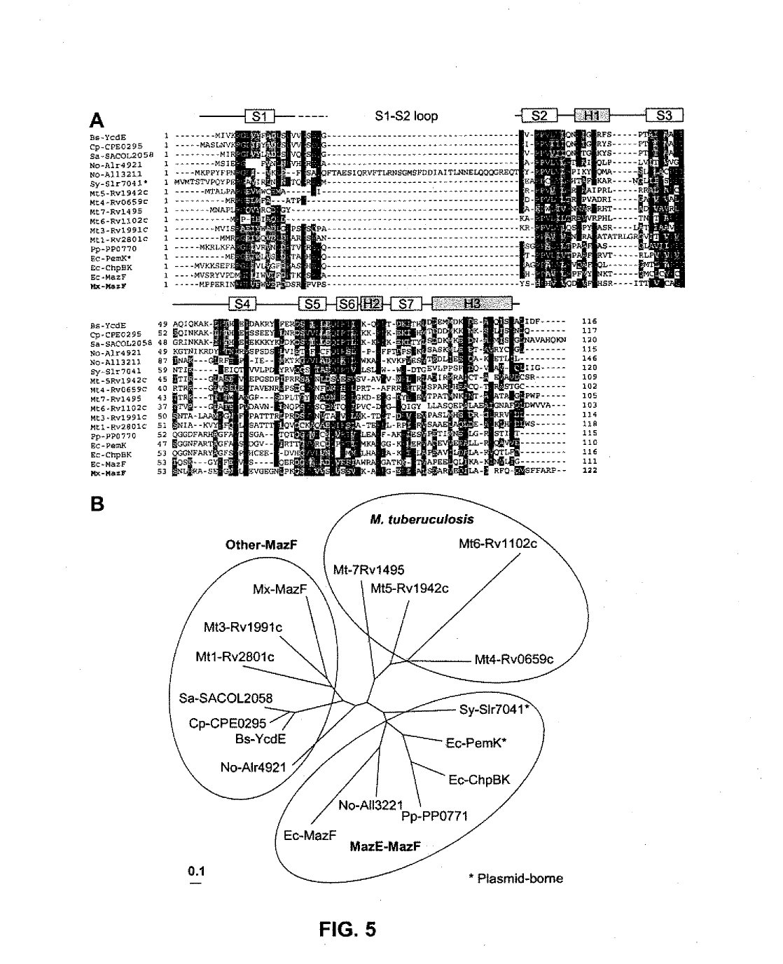

It was found that in contrast to all known MazE-MazF systems in a number of prokaryotes, M. xanthus MazF (MazF-mx) is encoded by a monocistoronic operon without any cognate antitoxin gene. Genomic analysis for the eleven known TA families using TBLASTN-Search, Pfam and COG lists on the M. xanthus genomic data-base (“TIGR”) revealed the existence of a single MazF homolog (MazF-mx; MAXN1659) with no identifiable MazE homolog (Table S1). MazF-mx (122 aa) has 24% identity and 58% similarity to E. coli MazF (111 aa) (FIG. 5A). The finding of such a solitary mazF gene appeared to be an exception to the hypothesis that the TA modules may play essential roles during adaptation to environmental stresses by inducing a state of reversible bacteriostasis (D. P. Pandey, K. Gerdes, Nucleic Acids Res. 33, 966 (2005)). It also raises intriguing questions as to whether MazF-mx expression may be developmentally regulated and associated with developmental autolysis, and if an antitoxin exists since MazF antitoxins are highly diverse (Table S2). Phylogenetic-tree analysis of MazF homologs (FIG. 5B) also suggests a diversity of MazF function as MazF homologs may be classified into several branches.

In order to identify the antitoxin for MazF-mx, a yeast two-hybrid screen was performed using MazF-mx as bait and an M. xanthus genomic library (H. Nariya, S. Inouye, Mol. Microbial. 56, 1314 (2005)). From 32 positive interactions found to associate with MazF-mx, 15 were mazF-mx and 17 were mrpC, indicating that MazF-mx forms an oligomer (dimer) and that MrpC may be a likely candidate antitoxin for MazF-mx.

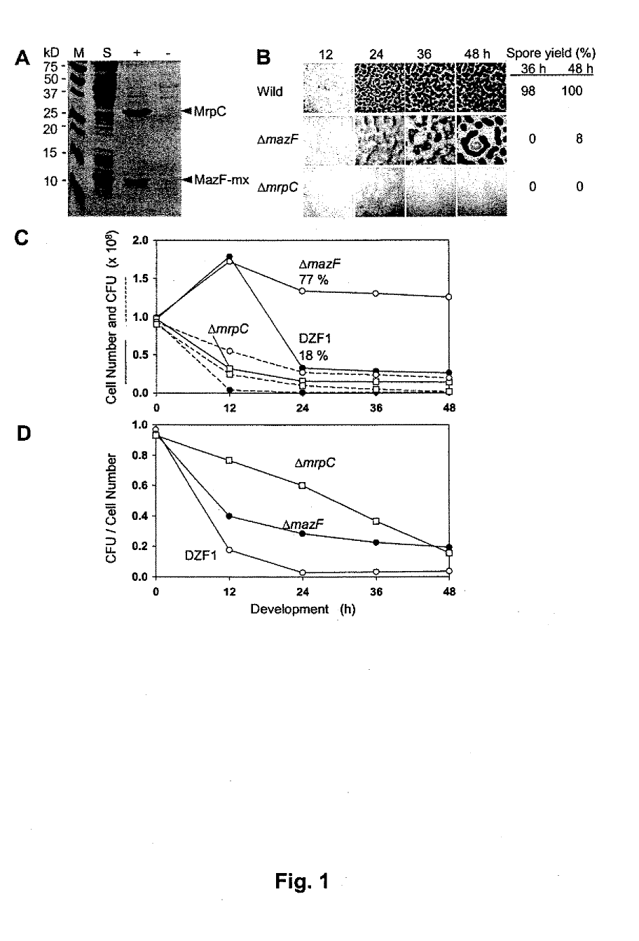

Interestingly, MrpC is a 248-residue protein, which is a member of the CRP transcription regulator family and is chromosomally located 4.44 Mbp downstream of the mazF-mx gene. Importantly, the mrpC gene is essential for M. xanthus development (H, Sun, W. Shi, J. Bacteriol. 183, 4786 (2001)), and is a key early-developmental transcription activator for the gene for FruA, another essential developmental regulator (T. Ueki, S. Inouye, Proc. Natl. Acad. Sci. U.S.A. 100, 8782 (2003)). Additionally phosphorylation of MrpC by a Ser/Thr kinase cascade is also involved in the regulation of MrpC function (H. Nariya, S. Inouye, Mol. Microbial. 60, 1205 (2006)). MrpC and MazF interaction can be further detected by pull-down assays using purified N-terminal histidine tagged MrpC and non-tagged MazF-mx expressed in the soluble fraction of E. coli (FIG. 1A).

In order to elucidate the role of MazF in the life cycle of M. xanthus, a mazF-mx in-frame deletion strain (ΔmazF) was constructed. While vegetative growth of ΔmazF was normal, it was observed that development was profoundly affected. When the concentrated vegetative cells at the mid-log phase (2×10¹⁰ cells/ml) of ΔmazF and the parental cells (DZF1) were spotted (5 μl; 10⁸ cells) onto limited-nutrient CF agar plate, DZF1 developed normally within 48 h forming compact fruiting bodies (“FB”) consisting of myxospores, while development of ΔmazF was delayed and compact FB were not formed producing very poor spore yields (at only 8% of the yield of wild-type spores; FIG. 1B). Even after 120 h of development, FB of ΔmazF cells appeared to be very loose and relatively translucent compare to DZF1. Cell autolysis and viability during development were also examined (FIG. 1C); cell numbers for both LmazF and DZF1 almost doubled cell numbers at 12 h after spotting on CF plates. After this time point, DZF1 cell numbers dramatically decreased to 18% due to autolysis. At the 24 h time point, the surviving wild-type cells begin to be converted to myxospores. In contrast, ΔmazF cell numbers only slightly reduced to 77% and were maintained at that level even at 48 h (FIG. 1C). Interestingly, DZF1 cell viability was substantially reduced (less than 1%) after 24 h of development, while over 30% of ΔmazF cells were able to form colonies on CYE plates (FIG. 1D). When development-defective ΔmrpC cells (H. Nariya, S. Inouye, Mol. Microbiol. 58, 367 (2005)) were examined in a similar manner, they were completely incapable of growth on CF plates (FIG. 1D), while cell viability only gradually decreased in contrast to DZF1 and ΔmazF (FIG. 1B). The ΔmrpC morphology on the starvation plates is shown in FIG. 1B, where no FB formation was observed and the cell viability continued to decrease (FIG. 1D). These observations indicate that MazF-mx is required for developmental autolysis to complete effective fruiting body formation and sporulation.

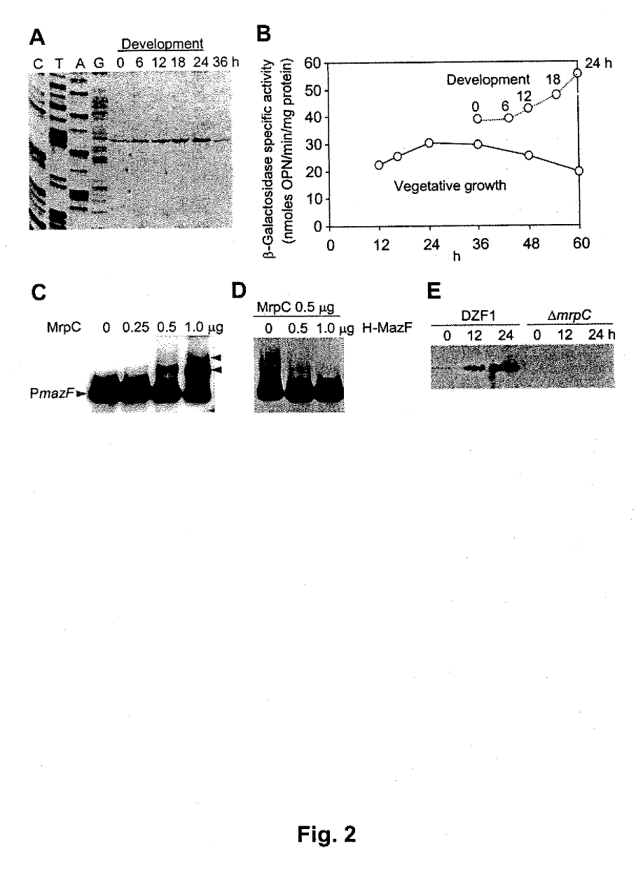

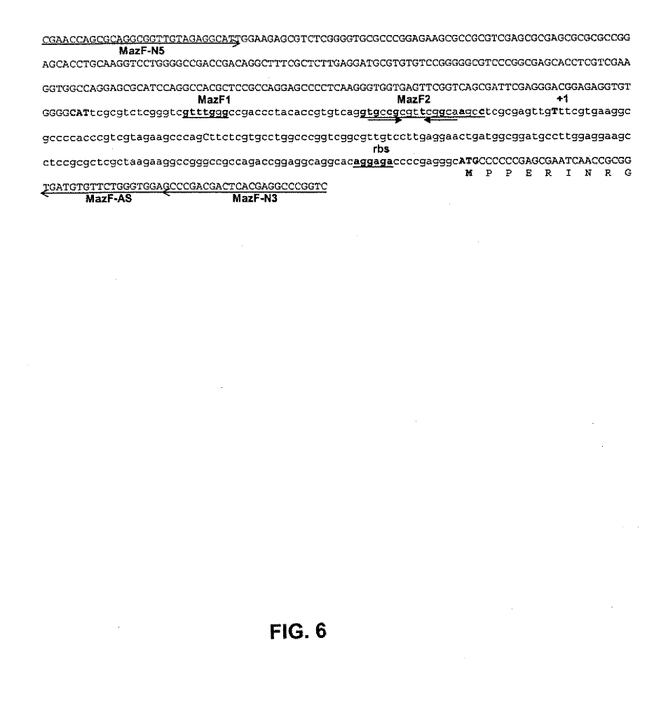

Since in E. coli, the expression of the mazEF operon is negatively auto-regulated by the MazE-MazF complex (I. Marianovsky, E. Aizenman, H. Engelberg-Kulka, G. Glaser, J. Biol. Chem. 276, 5975 (2001)), the role of MrpC in regulating mazF-mx expression was examined. By primer-extension (FIG. 2A) using total RNA isolated from DZF1, the transcriptional initiation site of mazF-mx was localized to 164-bases upstream from the initiation codon (FIG. 6) for both vegetative growth and the development phase. Notably, the level of mazF-mx transcript significantly increased upon nutritional starvation (FIG. 2A), indicating that mazF-mx is developmentally induced. To further confirm this notion, a lacZ-mazF-mx fusion was constructed and introduced into DZF1 at the original chromosomal location. β-galactosidase assay of this constructed strain (mazF-mx^{p}-lacZ/DZF1) showed that mazF-mx-lacZ was expressed at approximately 20˜30 U during vegetative growth and steadily increased after 6 h at the onset of development and reached 55 U at 24 h (FIG. 2B). These results are in agreement with the result of primer-extension analysis (FIGS. 2A and E).

Next examined was whether MrpC can bind to the mazF-mx promoter. Gel-shift assay using purified MrpC and the mazF-mx promoter region from −73 to +166 (PmazF; FIG. 2C) showed that MrpC binds to at least two sites on the mazF-mx promoter region. On the basis of the consensus sequence A/GTTTC/GAA/G and GTGTC-N₈-GACAC [N is any bases], two MrpC-binding sites may be assigned at the regions from −56 to −50 (MazF1) and from −29 to −12 (MazF2; FIG. 6). Binding of MrpC to the promoter region was found to be inhibited when MrpC was preincubated with MazF-mx (FIG. 2D). Furthermore, the mazF-mx expression in ΔmrpC, analyzed by primerextension (FIG. 2E), became undetectable during both vegetative growth and the development phase, indicating that MrpC is a transcription activator for developmental mazF-mx expression.

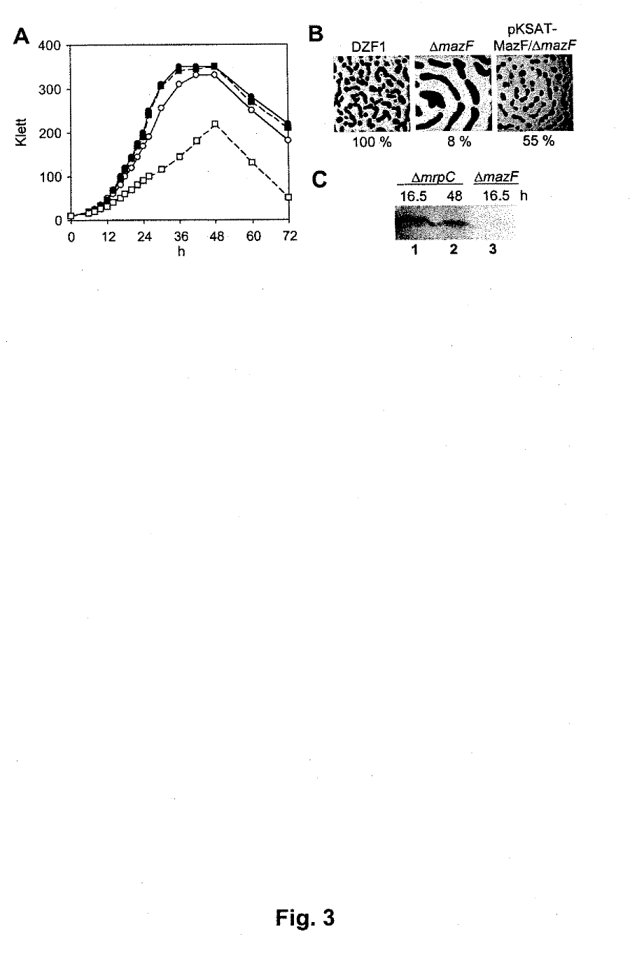

In order to detect MazF-mx toxicity in M. xanthus, mazF-mx was cloned in an M. xanthus expression vector, pKSAT, which can constitutively express a cloned gene during vegetative growth and the development phase. The resulting pKSAT-MazF-mx was then integrated into the chromosome by site-specific (attB/attP) recombination. Furthermore, a hemagglutinin epitope (HA)-tagged mazF-mx was also constructed and cloned in pKSAT (pKSAT-HA-MazF) to detect its expression in M. xanthus by Western blot analysis. These constructs were first introduced into ΔmazF, resulting in the strains, pKSAT/ΔmazF (vector control), pKSAT-MazF/ΔmazF and pKSAT-HA-MazF/ΔmazF. No significant growth defect was observed in any of the strains during vegetative growth (FIG. 3A). MrpC expression level in ΔmazF was similar to that in DZF1 during both vegetative growth and development. Importantly, the defective developmental phenotypes of ΔmazF were partially restored by the introduction of pKSAT-MazF, which could form compact FBs and yield myxospores at an intermediate level (FIG. 3B), while the introduction of pKSAT vector alone was unable to restore the phenotypes. Notably, severe cell-toxicity by MazF-mx was observed in ΔmrpC. While pKSAT-HA-MazF/ΔmrpC was able to grow in CYE medium, its growth-rate was significantly reduced and the cells could not reach to the maximum density (350 Klett) as the growth stopped at 220 Klett (FIG. 3A). Interestingly, the cells then rapidly lyzed forming aggregates (to 50 Klett), while the density of control cells only gradually decreased without forming aggregates (to 220 Klett) at 72 h. A very similar phenotype was observed with pKSAT-MazF/ΔmrpC, as cell viability of these cells was almost proportional to the Klett units. Expression of HA tagged MazF-mx in M. xanthus was confirmed by the Western blot analysis using an HA antibody at the mid-log and mid-stationary phase (FIG. 3C). These results indicate that MazF-mx expression in the absence of MrpC expression is toxic, confirming the prediction that MrpC functions as an antitoxin to MazF-mx.

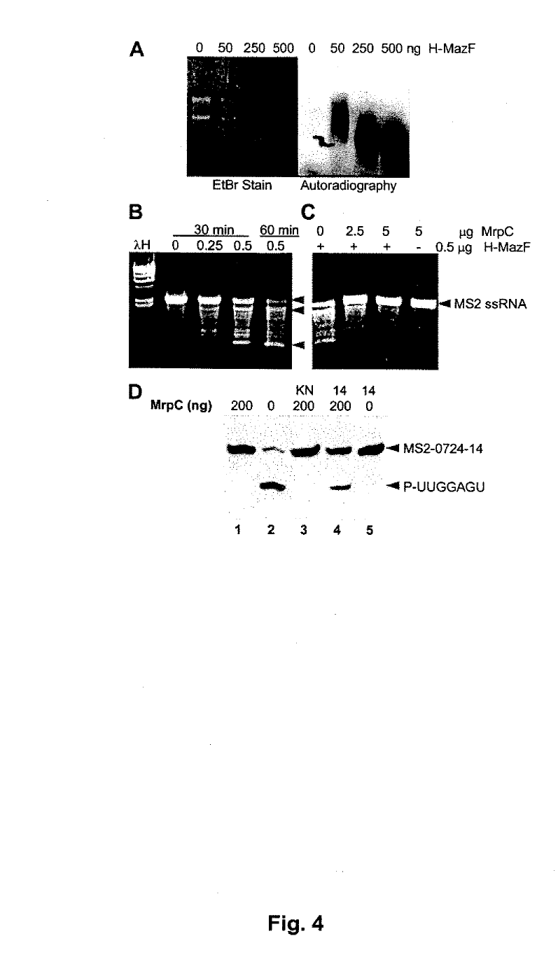

Since MazF-mx expression did not exhibit strong cellular toxicity in E. coli, MazF-mx may cleave mRNAs at a more specific site than E. coli MazF. Purified MazF-mx did show endoribonuclease activity yielding free 5′-OH group against M. xanthus total RNA (FIG. 4A). When MS2 phage ssRNA (3569-bases) was used as substrate, it was cleaved into major two bands of approximately 2.8 and 0.8-kb with many minor bands between them (FIG. 4B), suggesting that MS2 ssRNA may contain a preferential cleavage site for MazF-mx. Importantly preincubation of MazF-mx with MrpC almost completely inhibited the MazF-mx endoribonuclease activity (FIG. 4C), further demonstrating that MrpC functions as antitoxin for MazF-mx. Preliminary experiments of primer-extension analyses using a variety of primers and cleaved products have identified a preferential cleavage site on MS2 ssRNA, position 0724 (GAGU!UGCA; ! indicates the cleavage site), with a combination of other minor cleavage sites observed at high concentration of MazF-mx. Thus, MazF-mx appears to preferentially recognize the five base sequence, GU!UGC cleaving between U and U.

During vegetative growth, MrpC is reported to be phosphorylated by a eukaryotic-like Ser/Thr protein kinase cascade that suppresses MrpC function to prevent untimely switch-on of the early developmental pathway [Pkn8 (Pkn14 kinase)-Pkn14 (MrpC kinase) cascade; (H. Nariya, S. Inouye, Mol. Microbiol. 60, 1205 (2006))]. We, therefore, examined the effect of MrpC phosphorylation on the mRNA interferase activity of MazFmx, using a synthetic 14-base RNA substrate, MS2-0724-14 (UUGGAGU!UGCAGUU) that contains the consensus sequence for the most preferential cleavage site on MS2 ssRNA (FIG. 4D). When 50 ng of MazF-mx was preincubated with 200 ng of MrpC, MazF-mx activity on MS2-0724-14 completely inhibited (compare lane 1 with lane 2). However, when MrpC was incubated with Pkn14 in the presence of 1 mM ATP, the inhibitory function of MrpC was reduced (lane 4), while an autokinase-defect mutant, Pkn14K48N (H. Nariya, S. Inouye, Mol. Microbiol. 60, 1205 (2006)) could not affect the MrpC inhibitory function (lane 3). Note that Pkn14 by itself did not show RNase activity (lane 5). These results suggest that phosphorylation of MrpC by Pkn14 may block the inhibitory complex formation with MazF-mx. Note that the genetic disruption of the Pkn8-Pkn14 cascade causes up-regulation of mrpC resulting in acceleration of FB formation (H. Nariya, S. Inouye, Mol. Microbial. 60, 1205 (2006)).

Together, the findings disclosed herein reveal that M. xanthus has a PCD cascade that is developmentally regulated and composed of a Ser/Thr cascade (Pkn8-Pkn14), a developmental transcription factor/antitoxin (MrpC) and an mRNA interferase (MazF-mx). Upon the onset of FB formation, MrpC expression is induced, which then activates the transcription of the mazF-mx. Subsequent cleavage of cellular mRNAs by MazF-mx may cause further devastating metabolic effects to the cells whose growth is already severely inhibited by nutrition deprivation. This may trigger autolysis by inducing a number of autolytic enzymes. MrpC is a key regulator for activation of early developmental genes including mazF-mx. During early and middle development, MrpC is expressed at a high level (H. Nariya, S. Inouye, Mol. Microbiol. 60, 1205 (2006)) that likely is able to neutralize MazF-mx toxicity, while still up-regulating the mx-mazF expression. Before sporulation is initiated, MrpC is thought to be degraded by LonD and/or other unidentified cellular proteases, which then activates MazF-mx mRNA interferase function, resulting in developmental autolysis to provide nutrients for a minor population (20%) of cells, which are destined to form FB and subsequent myxospores. How the 20% population is selected to survive avoiding autolysis remains an intriguing question. Since M. xanthus development does not uniformly occur, the seemingly altruistic autolysis may be a matter of timing and the subpopulation in which the onset of the developmental program is delayed (may be because of their position in the cell cycle at the time of nutritional deprivation) may be retriggered by transient release of nutrition from autolyzed cells to initiate the late developmental process. In this selected population, MazF-mx function has to be subdued by the mechanism yet to be determined. It also remains to be elucidated if MazF-mx can trigger PCD through the cleavage of a specific mRNA(s) or rather does so by inflating a general damage to the cells as suggested in the case of E. coli MazF (H. Engelberg-Kulka, R. Hazan, S. Amitai, J. Cell. Sci. 118, 4327 (2005)). Thus the wildly prevailing toxin-antitoxin system in bacteria appears to have multiple-functions in bacterial physiology. These results demonstrate for the first time that solitary MazF has a distinct mission from those toxins encoded by an operon together with their cognate antitoxin, as it functions only for PCD (rather than cell growth arrest) in a sophisticated PCD cascade associating with protein Ser/Thr kinases, which is reminiscent to the eukaryotic PCD cascade.

### EXAMPLES

### Materials and Methods Bacteria, Growth Conditions, Plasmid and DNA Manipulation

M. xanthus FB (DZF1) (C. E. Morrison, D. R. Zusman, J. Bacterial. 140: 1036 (1979)) and its derivatives were cultured in CYE medium at 30° C. (J. M. Campos, J. Geisselsoder, D. R. Zusman, J. Mol. Biol. 119: 167 (1978)) supplemented with 80 μg/ml kanamycin or 250 μg/ml streptomycin when necessary. To initiate fruiting body development, M. xanthus cells were spotted on CF (D. C. Hagen, A. P. Bretscher, D. Kaiser, Dev. Biol. 64: 284 (1978)) and TM agar (H. Nariya, S. Inouye, Mol. Microbial. 49: 517 (2003)) plates and spore yields were measured as described previously (M. Inouye, S. Inouye, D. R. Zusman, Proc. Natl. Acad. Sci. U.S.A. 76: 209 (1979)). Autolysis during development was measured by counting cell numbers (H. Nariya, S. Inouye, Mol. Microbial. 49: 517 (2003)). Cell viability was examined by measuring colony formation units (CFU) plating cells on CYE plates. E. coli DH5α (D. Hanahan, J. Mol. Biol. 166: 557 (1983)) was used as the recipient strain for transformation and grown in LB medium (J. H. Miller, Experiments in Molecular Genetics. Cold Spring Harbor, N.Y.: Cold Spring Harbor Laboratory Press. (1972)) supplemented with 50 μg/ml kanamycin, 100 μg/ml ampicillin or 25 μg/ml streptomycin. E. coli BL21 (DE3) was used for the expression of mazF-mx under the control of a T7 promoter in a T7 vector (F. W. Studier, A. H. Rosenberg, J. J. Dunn, J. W. Dubendorff, Methods Enzymol. 185: 60 (1990)). The proteins were induced by the addition of 1 mM IPTG at 100 Klett (equivalent to 5×10⁸ cells/ml) in M9 medium (T. Maniatis, E. F. Fritsch, J. Sambrook, Molecular Cloning: A Laboratory Manual. Cold Spring Harbor, N.Y.: Cold Spring Harbor Laboratory Press. (1989)) supplemented with 100 μg/ml ampicillin. pUC19 (C. Yanisch-Perron, S. Vieira, J. Messing, Gene 33: 103 (1985)) was used to clone chromosomal DNA fragments. DNA sequences were determined by an ABI Genetic Analyzer 310 using the methods provided by the company and double-stranded plasmid DNA as templates. M. xanthus genomic DNA was used as template for PCR amplification. PCR-amplified regions were confirmed by DNA sequencing. Other DNA manipulations were carried out by the methods described previously (J. Munoz-Dorado, S. Inouye, M. Inouye, Cell 67: 995 (1991)).
   Construction of a mazF-mx in-Frame Deletion Strain, ΔmazF and a mazF-mx-lacZ-Fusion Strain

A method developed based on the cell toxicity by galK (galactokinase gene) (T. Ueki, S. Inouye, M. Inouye, Gene 183: 153 (1996)) was used for construction of an in frame deletion of MazF-mx between Pro-24 to Ser-100 (FIG. 5A). Since the genomic data-base for M. xanthus (http://cmr.tigr.org/tigr-scripts/CMR/GenomePage.cgi?org=gmx) shows that M. xanthus does not contain galK and galT (galactose-1-phosphate uridylyltransferase gene), D-(+)-galactose can be used in this system in place of 2-deoxygalactose. Two PCR fragments (MazF-N (SEQ ID NO. 11); 577-bp and MazF-C (SEQ ID NO. 12); 566-bp) amplified using the M. xanthus chromosomal DNA as template by the following primers; one fragment with MazF-N5 (AAAGAATTCAAGCTTCGAACCAGCGCAGGCGGTTGTAGAGGCAT) (SEQ ID NO. 1) and MazF-N3 (AAAGGATCCAAAGTCGACCGGGCCTCGTGAGTCGTCGGGCTCCA) (SEQ ID NO. 2), and the other fragment with MazF-05 (AAAGAATTCAAGCTTGTCGACGCGCGGGTGGAACAGATTCTTGCC) (SEQ ID NO. 3) and MazF-C3 (AAAGGATCCTCAAGACGAGCCCGCCAGCGAAGAGCACT) (SEQ ID NO. 4). These fragments were cloned into pKO1Km^{R} (T. Ueki, S. Inouye, M. Inouye, Gene 183: 153 (1996)) at EcoRI and BamHI sites resulting in plasmids, pMazF-N and pMazF-C, respectively. The SalI-BamHI fragment from pC-MazF were inserted into pMazF-N at Sal I-BamHI, resulting in pMazF-IF, which has an in-frame fusion between Val23 (GTC) and Asp101 (GAC). pMazF-IF was electroporated into DZF1 cells for single crossing-over recombination (1st recombination) to screen kanamysin-resistant cells on CYE plates containing 80 μg/ml kanamycin. Kanamycin-resistant colonies were then subjected to colony-directed PCR to determine the sites of integration, using following primers; for upstream integration (N-cross), MazF-5 (GTGGGCGCGAAGTGCGCAGCCGTGTCT) (SEQ ID NO. 5) and Km-1 (CTGGCTTTCTACGTGTTCCGCTTCCTTTAGC) (SEQ ID NO. 6) in pKO1Km^{r}, and for downstream integration (C-cross), MazF-5 (SEQ ID NO. 5) and MazF-IC (TCGTCGTCGTGTCGCAGGTGTCCTCGGT) (SEQ ID NO. 7). N- and C-cross strains identified above were individually cultured in CYE medium to 100 Klett, and then serially diluted cultures with CYE medium were plated on CYE agar plates containing 10 mg/ml D-(+)-galactose (Sigma). Kanamycin-sensitive and galactose-resistant colonies resulted from the second recombination looping out the plasmid-derived region were either the original wild-type, DZF1 or the in-frame deletion strain (ΔmazF). The ΔmazF mutation was identified by colony-directed PCR using two sets of primers; one with MazF-5 (SEQ ID NO. 5) and MazF-I (GAGTGATTGAAGACGTCGTCCTGAACCACCA) (SEQ ID NO. 8) and the other with MazF-5 (SEQ ID NO. 5) and MazF-C3 (SEQ ID NO. 4). Since the phenotype during vegetative growth and development of both ΔmazF strains obtained from both N- and C-cross was identical, they were used as ΔmazF.

The lacZ-fusion strain with the mazF-mx promoter region was constructed by insetting MazF-N (SEQ ID NO. 11) fragment (−344 to +233) digested with HindIIII and BamHI into pZK (H. Nariya, S. Inouye, Mol. Microbial. 56, 1314 (2005)), resulting in pZK-mazF^{p}. β-galactosidase assays were carried out as described previously (H. Nariya, S. Inouye, Mol. Microbial. 56, 1314 (2005), L. Kroos, A. Kuspa, D. Kaiser, Dev. Biol. 117: 252 (1986)).

### Primer-Extension Analysis

Total RNA was isolated by the hot-phenol method from DZF1 and ΔmrpC cells grown in CYE medium harvested at the early-log (12 h/50 Klett), mid-log (16.5 h/100 Klett), late-log (24 h/200 Klett), early-stationary (36 h/350 Klett), mid-stationary (48 h/350 Klett) and late-stationary (60 h/280 Klett) phases (H. Nariya, S. Inouye, Mol. Microbial. 56, 1314 (2005)). The early-stationary phase cells were spotted on TM agar plates to initiate fruiting body development, and developmental cells were collected at 0, 6, 12 and 24 h as described previously (H. Nariya, S. Inouye, Mol. Microbial. 56, 1314 (2005)). Primer-extension was carried out using primer MazF-AS (FIG. 6) as described previously (H. Nariya, S. Inouye, Mol. Microbial. 49: 517 (2003)). The extended products were analyzed on a 6% polyacrylamide gel containing 8 M urea and a sequencing ladder was made with the same primer using pMazF-N as template (FIG. 2A).

Construction of M. xanthus Expression Vector, pKSAT

Since the kanamycin resistance gene (km^{r}) from Tn5 is generally used as a drug-marker in M. xanthus and known to be constitutively expressed during both vegetative growth and development, its promoter region (159-bp) was amplified by PCR with primers, Km-P5 (AAAGGTACCACAGCAAGCGAACCGGAATTGCCA) (SEQ ID NO. 9) and Km-P3 (AAACATATGAAACGATCCTCATCCTGTCTC) (SEQ ID NO. 10) using pUC7Km(P−) as template (N. Norioka, M. Y. Hsu, S. Inouye, M. Inouye, J. Bacterial. 177: 4179 (1995)). The resulting DNA fragment was cloned into pBluescript II SK(−) (Stratagene) between KpnI and NdeI sites, resulting in pKA. The 1.9-kbp NdeI-HincII fragment containing strA-strB genes from Salmonella typhimurium plasmid R64 (T. Komano, T. Yoshida, K. Narahara, N. Furuya, Mol. Microbial. 35: 1348 (2000)) was then inserted between two SspI sites in pKA, resulting in pKS. For attB/attP recombination in M. xanthus, the 2.9-kbp SmaI fragment containing intP-attP from Myxophage M×8 (N. Tojo, K. Sanmiya, H. Sugawara, S. Inouye, T. Komano, J. Bacterial. 178: 4004 (1996)) was inserted between two DraI sites, resulting in pKSAT. In this plasmid, the transcription directions of both strA-strB and intP-attP were selected to be the same as that of the km^{r} promoter. pKSAT contains NdeI and BamHI sites for cloning genes for expression.

### Yeast Two-Hybrid Screen for Identification of the Antitoxin for MazF-mx

The 0.4-kb NdeI-BamHI fragment from mazF-mx was amplified by PCR using primers; MazF-N (AAACATATGCCCCCCGAGCGAATCAACCGCGGTGA) (SEQ ID NO. 11) and MazF-C (AAAGGATCCTCACGGCCTCGCGAAGAACGACACCTGCT) (SEQ ID NO. 12), and cloned into pGBD-NdeI for bait and pGAD-NdeI for target to perform a yeast two-hybrid screen (H. Nariya, S. Inouye, Mol. Microbial. 56, 1314 (2005)). The yeast strain PJ69-4A was used for the yeast two-hybrid screen (P. James, J. Halladay, E. A. Craig, Genetics 144: 1425 (1996)) and the M. xanthus genomic DNA library used is described previously (H. Nariya, S. Inouye, Mol. Microbial. 56, 1314 (2005)). Interaction between MazF-mx and MrpC in the yeast two-hybrid screen was examined by quantitative β-galactosidase activity assay (H. Nariya, S. Inouye, Mol. Microbial. 56, 1314 (2005)). MrpC and MazF-mx interact at a level of 5.0 U while MazF-mx/MazF-mx interaction is strong at a level of 42.5 U (control is 0.3 U),

### Expression and Purification of MazF-mx

The mazF-mx fragment was also cloned into pET-11a and pET-16b(+) (Novagene) resulting in pET-MazF or pET-H-MazF, respectively. Both non-tagged MazF-mx and N-terminal histidine-tagged MazF-mx (H-MazF) induced in E. coli BL21 (DE3) by IPTG for 3 h were soluble. H-MazF was purified using Ni-NTA SUPER FLOW resin (Qiagen) as described before (H. Nariya, S. Inouye, Mol. Microbial. 58, 367 (2005)). The eluted fraction from the resin was then dialyzed against 50 mM Tris-HCl, pH 8.0 containing 20% (w/v) glycerol, followed by passing through HiTrap SP and Q columns (GE). H-MazF was recovered from the flow-through pool by the resin. The eluted fraction was dialyzed against MazF buffer [25 mM Tris-HCl, pH 8.0 containing 100 mM NaCl, 5% (w/v) glycerol and 0.5 mM DTT], and purified H-MazF (0.5 mg/ml) was stored at −80° C. Gel filtration analysis using purified H-MazF (200 μl) was performed as described previously (H. Nariya, S. Inouye, Mol. Microbiol. 58, 367 (2005)). H-MazF (15.9 kD on SDS-PAGE) was eluted at the position of ˜30 kD (dimer).

Interaction of MazF-mx with MrpC

A pull-down assay was carried out as previously described (H. Nariya, S. Inouye, Mol. Microbiol. 56, 1314 (2005)). 500 μl of crude soluble fraction (S) from E. coli (2000 Klett/ml) expressing non-tagged MazF-mx was incubated with (+) or without (−) 25 μg of purified N-terminal histidine-tagged MrpC (H. Nariya, S. Inouye, Mol. Microbiol. 58, 367 (2005)). The complex was recovered by 10 μl of the Ni-NTA resin (FIG. 1A). The complex thus formed was analyzed by SDS-PAGE.

Expression of MazF-mx in M. xanthus

Hemagglutinin epitope (HA)-tagged mazF-mx was amplified by PCR using primers, MazF-HA (AAACATATGGGGTACCCCTACGACGTGCCCGACTACGCCATGCCCCCCGAGC GAATCA ACCGCGGTGA) (SEQ ID NO. 13) and MazF-C (SEQ ID NO. 12). The HA-tagged and non-tagged mazF-mx genes were then cloned into pKSAT at NdeI and BamHI sites resulting in plasmids, pKSAT-MazF and pKSAT-HA-MazF, respectively. They were integrated into the chromosome of ΔmazF and ΔmrpC by site-specific (attB/attP) recombination (H. Nariya, S. Inouye, Mol. Microbiol. 49: 517 (2003)) resulting in strains, pKSAT-HA-MazF/ΔmrpC, and pKSAT-MazF/ΔmazF, respectively. pKSAT was also integrated into ΔmazF and ΔmrpC strains, resulting in strains, pKSAT/ΔmazF and pKSAT/ΔmrpC, respectively.

Expression of MazF-mx in ΔmrpC (10⁸ cells) carrying pKSAT-HA-MazF during vegetative growth was detected by Western blot using HA antibody.

### Gel-Shift Assay

The promoter region of mazF-mx (PmazF: −73 to +166) was amplified by PCR using primers, MazF-N5 (SEQ ID NO. 1) and MazF-N3 (SEQ ID NO. 2) (FIG. 6). The product was purified by agarose gel electrophoresis using the QIAquick Gel Extraction Kit (Qiagen). Purified PmazF was then labeled at the 5′ end with [γ-³²P]-ATP by T4 kinase (Invitrogen), followed by further purification using the QIAquick PCR purification Kit (Qiagen). The gel-shift assay (FIGS. 2C and 2D) was carried out using purified MrpC and labeled PmazF (10 finales) as described previously (H. Nariya, S. Inouye, Mol. Microbial. 60, 1205 (2006)). MrpC was incubated with H-MazF in 5 μl of MazF buffer for 10 min at 30° C., and subjected to the gel-shift assay (FIG. 2D).

mRNA Interferase Activity of MazF-mx

M. xanthus total RNA isolated from mid-log cells was treated with 1 mM ATP and T4 kinase on ice for 60 min to mask all the free 5′ ends, and purified on a Qiagen column using PB and PE buffer (Qiagen). Purified RNA (0.1 μg) was digested with H-MazF in 20 μl of MazF buffer for 30 min at 30° C. Products were then labeled with [γ-³³P]-ATP by T4 kinase. Denatured products in urea were separated on an 1.2% TBE native agarose gel (Y. C. Liu, Y. C. Chou, Biotechniques 9: 558 (1990)). The gel was stained with ethidium bromide (EtBr) and then dried with a gel drier. The dried gel was subjected to autoradiography (FIG. 4A).

MS2 ssRNA (0.8 μg; 3569-bases; Roche) was digested by H-MazF in 20 μl of MazF buffer at 30° C. as indicated (FIG. 4B). H-MazF was preincubated with MrpC for 10 min, and then further incubated with MS2 ssRNA for 30 min (FIG. 4C).

MrpC (2.5 μg) was incubated with 10 μg of Pkn14 or autokinase-defect mutant, Pkn14K48N (KN) (H. Nariya, S. Inouye, Mol. Microbial. 60, 1205 (2006)) in 50 μl of P buffer with 1 mM ATP at 30° C. for 4 h, followed by dialysis against MazF buffer containing 200 mM NaCl at 4° C. 4 μl (200 ng MrpC) of dialysates were preincubated with H-MazF (50 ng) in 20 μl of MazF buffer for 10 min at 30° C. To this solution, 0.01 pmole of 5′-end γ-³²P labeled MS2-0724-14 (a 14-base synthetic RNA substrate; see the text) was added and the mixture was for 30 min at 30° C. For control, MS0724-14 was incubated with only Pkn14. Reactions were stopped by addition of 12 μl of sequencing loading buffer (Stop Solution; Roche) and heated at 95° C. for 2 min and then placed on ice. The product was separated by 20% TBE-PAGE and the gel was subjected to autoradiography (FIG. 4D).

TABLE S1
Chromosomal TA modules in spore-forming bacteria

| Organism/TA family ^{a} | MazEF | RelBE | ParDE | HigBA | VapBC | Phd/Doc | CcdAB | Total |
|---|---|---|---|---|---|---|---|---|
| B. subtilis 168 | 1 | 0 | 0 | 0 | 0 | 0 | 0 | 1 |
| B. anthracis | 1 | 0 | 0 | 0 | 0 | 0 | 0 | 1 |
| C. perfringens 13 | 1 | 0 | 0 | 0 | 0 | 1D | 0 | 2 |
| C. acetobutylicum | 1 | 0 | 0 | 0 | 0 | 0 | 0 | 1 |
| S. coelicolor 3A(2) | 0 | 1 | 0 | 0 | 0 | 2 | 0 | 3 |
| S. avermitilis MA | 1F | 1 | 0 | 0 | 1 | 2 | 0 | 5 |
| M. xanthus DK1622 | 1F | 0 | 0 | 0 | 0 | 0 | 0 | 1 |

^{a} Genomic survey of the seven known TA families was examined by Pandy and Gerdes (2005) except for that of M. xanthus in this study. 1F and 1D indicate solitary MazF and Doc, respectively.

TABLE S2
Diversity of antitoxin for MazF

| Organism | MazEF | MazE/Antitoxin ^{a} | bp ^{b} | MazF ^{c} |
|---|---|---|---|---|
| E. coli K12 | 2 | MazE (b2783 82 aa) | −1 | MazF (b2782 111 aa) |
| | | ChpBI (b4224 85 aa) | −7 | ChpBK (b4225 116 aa) |
| P. putida KT244 | 1 | PP0770 (84 aa) | −4 | PP0771 (115 aa) |
| P. aeruginosa PAO1 | 0 | NF | | NF |
| B. subtilis 168 | 1 | CopG (YcdD 93 aa) | +4 | YcdE (116 aa) |
| C. perfringens 13 | 1 | CopG (NA 80 aa) | +5 | CPE0295 (117 aa) |
| S. aureus COL | 1 | Unk (SACOL2059 56 aa) | −4 | SACOL2058 (120 aa) |
| Synechocystis PCC6803 | 1 | Unk (Ssl2245 88 aa) | −4 | Sll1130 (115 aa) * |
| Nostoc PCC7120 | 4 + 1F | Asl3212 (80 aa) | −1 | All3211 (146 aa) |
| | | Unk (Asr4920 80 aa) | +5 | Alr4921 (115 aa) |
| | | Unk (Asl0338 61 aa) | −20 | All0337 (121 aa) * |
| | | Unk (Asr0757 69 aa) | −14 | Alr0758 (113 aa) * |
| | | NF | | Asr3006 (88 aa) * |
| M. tuberculosis H37Rv | 9 | Unk (NA 76 aa) | −13 | Rv2801c (118 aa) Mt1 |
| | | Unk NA 57 aa) | −11 | Rv0456A (93 aa) Mt2 * |
| | | Unk (NA 92 aa) | −4 | Rv1991c (114 aa) Mt3 |
| | | Unk (Rv0660c 81 aa) | −14 | Rv0659c (102 aa) Mt4 |
| | | Unk (Rv1943c 78 aa) | −4 | Rv1942c (109 aa) Mt5 |
| | | Unk (Rv1103c 78 aa) | −1 | Rv1102c (103 aa) Mt6 |
| | | Unk (Rv1494 100 aa) | −4 | Rv1495 (105 aa) Mt7 |
| | | Unk (NA 82 aa) | +31 | Rv2274c (105 aa) Mt8 * |
| | | Unk (Rv2063 77 aa) | −5 | NA (136 aa) Mt9 |
| S. coelicolor 3A(2) | 0 | NF | | NF |
| S. avermitilis MA-4680 | 1F | NF | | SAV671 (158 aa) * |
| M. xanthus DK1622 | 1F | NF | | MAXN1659 (122 aa) |

^{a} Those which have high homology to MazE are indicated in bold, and all the other unknown presumed antitoxins are indicated by Unk, NF and NA indicate those not found and not assigned in their genomics, respectively.
^{b} Distance between the antitoxin and MazF gene.
^{c} Asterisk indicates ORF displaying a weak similarity to MazF or having truncation (Asr3006 and Rv0456A).

What is claimed:

1. A method of regulating MazF-mx endoribonuclease activity comprising contacting MazF-mx with MrpC.

2. The method of claim 1, wherein MrpC is used as an antitoxin for MazF-mx.

3. A method of inhibiting the development of Myxococcus xanthus comprising inactivating the mazF-mx gene.

4. The method of claim 3, wherein the spore formation of Myxococcus xanthus is reduced.

5. The method of claim 3, wherein cell lysis of Myxococcus xanthus is inhibited.

6. The method of claim 3, wherein the mazF-mx gene is inactivated by MrpC.

7. An isolated MazF-mx polypeptide as disclosed herein.

8. A polypeptide having an amino acid sequence which has 90% identity with the amino acid sequence of the polypeptide of claim 7 and having endoribonuclease activity.

9. A polynucleotide encoding the MazF-mx polypeptide of claim 7.

10. The polynucleotide according to claim 9 having a nucleotide sequence as disclosed herein.

11. A polynucleotide which hybridizes to the complement strand of the polynucleotide of claim 10 in stringent conditions.

12. (canceled)

13. The polynucleotide of claim 9, comprising a promoter region having a DNA sequence as disclosed in FIG. 6.

14. The polypeptide of claim 7, wherein the polypeptide cleaves RNA at GUUGC.

15. The polypeptide of claim 7, wherein the polypeptide cleaves RNA at GUUGC between the two U residues.


SEQUENCE LISTING (13 sequences)

SEQ ID NO: 1 - DNA, 44 bp, Myxococcus xanthus
aaagaattcaagcttcgaaccagcgcaggcggttgtagaggcat

SEQ ID NO: 2 - DNA, 44 bp, Myxococcus xanthus
aaaggatccaaagtcgaccgggcctcgtgagtcgtcgggctcca

SEQ ID NO: 3 - DNA, 45 bp, Myxococcus xanthus
aaagaattcaagcttgtcgacgcgcgggtggaacagattcttgcc

SEQ ID NO: 4 - DNA, 38 bp, Myxococcus xanthus
aaaggatcctcaagacgagcccgccagcgaagagcact

SEQ ID NO: 5 - DNA, 27 bp, Myxococcus xanthus
gtgggcgcgaagtgcgcagccgtgtct

SEQ ID NO: 6 - DNA, 31 bp, Myxococcus xanthus
ctggctttctacgtgttccgcttcctttagc

SEQ ID NO: 7 - DNA, 28 bp, Myxococcus xanthus
tcgtcgtcgtgtcgcaggtgtcctcggt

SEQ ID NO: 8 - DNA, 31 bp, Myxococcus xanthus
gagtgattgaagacgtcgtcctgaaccacca

SEQ ID NO: 9 - DNA, 33 bp, Artificial Sequence
  Primer used to amplify promoter region of Tn5
aaaggtaccacagcaagcgaaccggaattgcca

SEQ ID NO: 10 - DNA, 30 bp, Artificial Sequence
  Primer used to amplify promoter region of Tn5
aaacatatgaaacgatcctcatcctgtctc

SEQ ID NO: 11 - DNA, 35 bp, Myxococcus xanthus
aaacatatgccccccgagcgaatcaaccgcggtga

SEQ ID NO: 12 - DNA, 38 bp, Myxococcus xanthus
aaaggatcctcacggcctcgcgaagaacgacacctgct

SEQ ID NO: 13 - DNA, 68 bp, Myxococcus xanthus
aaacatatggggtacccctacgacgtgcccgactacgccatgccccccgagcgaatcaaccgcggtga